(19) United States
(12) Patent Application Publication
(10) Pub. No.: US 2005/0113750 A1
(43) Pub. Date: May 26, 2005

(54) Safety needle

(76) Inventor: David John Targell, Ayrshire (GB)

Correspondence Address: BAKER & DANIELS;111 E. WAYNE STREET, SUITE 800, FORT WAYNE, IN, 46802, US

(21) Appl. No.: 10/751,384

(22) Filed: Jan. 5, 2004

(30) Foreign Application Priority Data
Nov. 21, 2003 (GB) GB 0327136.8

(51) Int. Cl. A61M 5/00

(52) U.S. Cl. 604/110; 604/263

(57) ABSTRACT

A safety needle (20) for use with a syringe including a casing (30) in which a needle (28) for injections or blood drawing is mounted. A sleeve (22) is slidably mounted in the casing (30). The sleeve (22) is biased by a spring (42) into a partially extended position, exposing only the sharp tip (26) of the needle. As the needle (28) is inserted into a patient, the sleeve (22) retracts into the casing. When the needle (28) is removed, the sleeve (22) returns to a fully extended position and by operation of a locking mechanism locks into place. The locking mechanism includes a plurality of fingers (62), at least one of which has a projection (64) formed thereon. The projection (64) travels along a track system (66) integrally formed in the inner wall (56) of the casing (30) to move from the initial sleeve position to the retracted position and then back to a locked position in which contact with the used needle (28) and reuse of the safety needle (20) is prevented.

### BACKGROUND OF THE INVENTION

1. Field of the Invention

The present invention relates to an improved safety needle for use with a syringe and accessories therefor.

2. Description of the Related Art

In the medical industry, injection devices such as syringes and needles are used everyday. Safety precautions should be taken to prevent the user, phlebotomist, nurse, doctor, or medical technician from being stuck with a used needle and potentially transferring blood-related diseases. Further, precautions must be taken to eliminate use of the same needle more than one time.

The prior art has provided safety needles which include a slidable sleeve positioned in surrounding relationship of the needle such as those described in U.S. Pat. Nos. 4,813,940 and 5,104,384, assigned to the assignee of the present invention. A sleeve covers the shaft of the needle, with the sharp needle tip exposed prior to use. As the needle is inserted into the patient, the sleeve makes contact with the patient and retracts into the safety needle casing against the bias of a spring. As the needle is removed from the patient, the sleeve remains in contact with the patient until the needle is completely removed and then the sleeve returns to its fully extended position, covering the used tip of the needle. The end of the sleeve is generally tapered toward an opening having a diameter small enough to prevent a person from contacting with the needle with a finger for example. Once the sleeve is in the fully extended position after use of the syringe, the safety needle is provided with means for locking the sleeve in that position.

One particular prior art means for locking the sleeve position after use of the safety needle includes a collar mounted in the safety needle casing. The sleeve is provided with a projection that engages a track formed in the collar. As the sleeve moves into the casing, the projection follows the pathway defined by the track such that, as the sleeve returns to its extended position, the projection is directed into a locking mechanism. Once the projection is received in the locking mechanism, the sleeve is prevented from retracting and exposing the used needle.

A problem with this method of locking the extended position of the sleeve is that, after use of the device, the track and the locking mechanism formed in the collar are fragile and may easily be damaged to allow the sleeve to retract and to again expose the needle. Further, due to the small size of the safety needle, there are difficulties molding the collar. The safety needle may also be difficult to assemble due to the small and fragile nature of the parts.

It is desired to provide a safety needle having a more robust design with an improved method of locking the slidable sleeve in an extended position.

### SUMMARY OF THE INVENTION

The present invention provides a safety needle for use with a syringe having a sleeve for preventing use of or being stuck with a used needle.

The present invention includes a casing in which a needle for use, for instance, with a hypodermic syringe or phlebotomy device is mounted. A sleeve is slidably mounted in the casing. The sleeve is biased by a spring into a partially extended position in which the sharp end of the needle is exposed, but the main shaft of the needle is encased by the sleeve. As the needle is inserted into a patient, the end of the sleeve makes contact with the patient. The sleeve retracts against the bias of the spring into the casing and remains in contact with the patient while the needle is being inserted, thus preventing exposure of the needle. When the needle is removed, the sleeve returns to a fully extended position, covering the contaminated needle tip, and, by operation of a locking mechanism, locks into place, thus preventing contact with the used needle and reuse of the needle.

The sleeve is provided with a plurality of fingers, at least one of which has a projection formed thereon. The projection engages a track system integrally formed in the inner wall of the safety needle casing. The projection is received in a recessed cavity formed in the casing wall in the initial position of the sleeve. As the sleeve retracts, the projection travels into a longitudinal passageway formed in the wall. When the needle is removed from the patient, the sleeve extends with the projection passing through the passageway. The projection is directed by a cam profile formed in the track system into a second passage portion. The projection exits the passageway and the fingers engage an annular ledge formed in the casing to lock the sleeve in an extended position and to prevent contact with the needle.

An advantage of the present invention is that the safety needle is easy to assemble, including fewer parts with less fragile components than prior art devices.

A further advantage of the present invention is that the safety needle includes an improved and simplified locking mechanism without fragile components.

An additional advantage of the present invention is that the safety needle has a more robust design which reduces the difficulty of assembly and reduces the possibility of the locking mechanism breaking which would allow the sleeve to again retract after the needle has been used.

### BRIEF DESCRIPTION OF THE DRAWINGS

The above mentioned and other features and objects of this invention, and the manner of attaining them, will become more apparent and the invention itself will be better understood by reference to the following description of embodiments of the invention taken in conjunction with the accompanying drawings, wherein:

FIG. 1 is a perspective view of a safety needle in accordance with the present invention with the sleeve in a partially extended position;

FIG. 2 is a perspective view of the needle of FIG. 1 with the sleeve in a retracted position;

FIG. 3 is a perspective view of the needle of FIG. 1 with the sleeve in a fully extended, locked position;

FIG. 4 is a sectional view of the needle of FIG. 1 taken along line 4-4;

FIG. 5 is a sectional view of the needle of FIG. 2 taken along line 5-5;

FIG. 6 is a sectional view of the needle of FIG. 3 taken along line 6-6;

FIG. 7 is an exploded perspective view of the needle of FIG. 1;

FIG. 8 is a sectional view of the needle casing of FIG. 7 taken along line 8-8;

FIG. 9 is a sectional view of the needle casing of FIG. 7 taken along line 9-9;

FIG. 10 is a sectional view of the needle casing of FIG. 7 taken along line 10-10;

FIG. 11 is a sectional view of the needle casing of FIG. 7 taken along line 11-11;

FIG. 12 is a fragmentary perspective view of a section of the needle casing of FIG. 7 taken along line 12-12;

FIG. 13 is a fragmentary perspective view of a section of the needle casing of FIG. 12 showing the locking fingers in an installed, initial position;

FIG. 14 is a fragmentary perspective view of a section of the needle casing of FIG. 12 showing the locking fingers moving from the initial position as the sleeve retracts;

FIG. 15 is a fragmentary perspective view of a section of the needle casing of FIG. 12 showing the locking fingers in a position in which the sleeve is completely retracted;

FIG. 16 is a fragmentary perspective view of a section of the needle casing of FIG. 12 showing the locking fingers moved toward the locked position as the sleeve extends; and

FIG. 17 is a fragmentary perspective view of a section of the needle casing of FIG. 12 showing the locking fingers in a locked position when the sleeve is fully extended.

Corresponding reference characters indicate corresponding parts throughout the several views. Although the exemplification set out herein illustrates several embodiments of the invention, in one form, the embodiments disclosed below are not intended to be exhaustive or to be construed as limiting the scope of the invention to the precise forms disclosed.

### DESCRIPTION OF THE PRESENT INVENTION

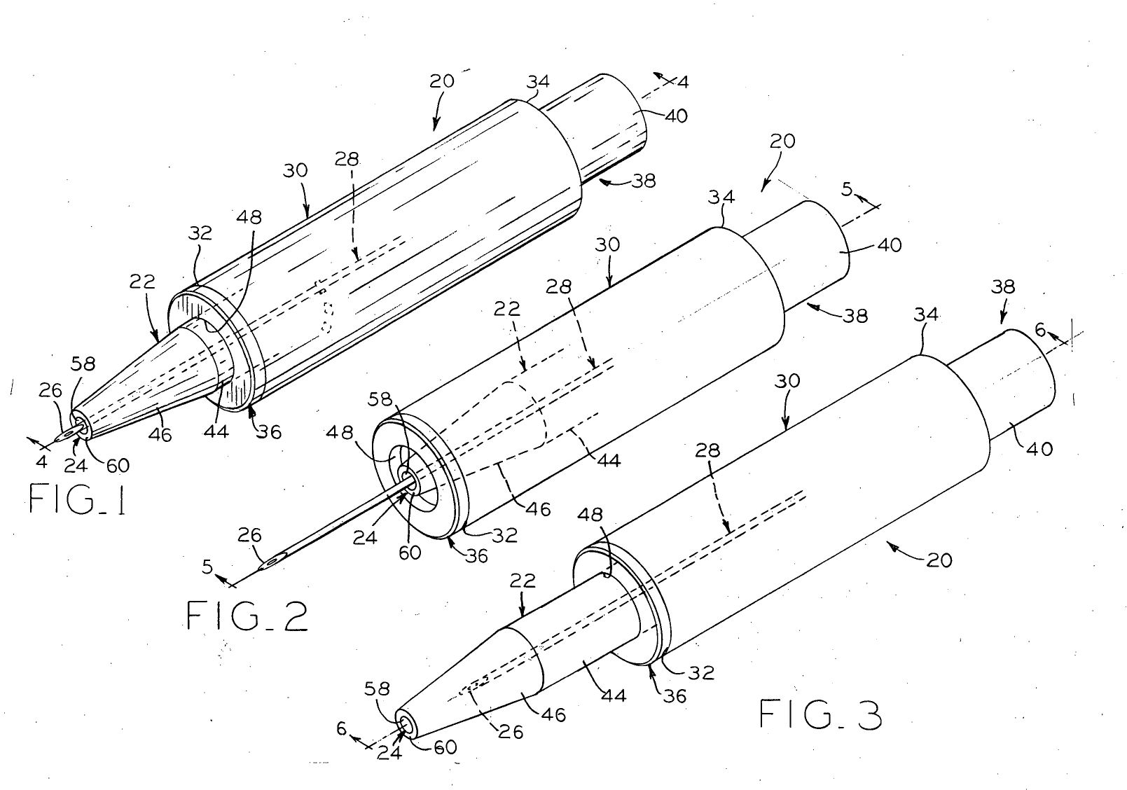

Referring to FIGS. 1, 2 and 3, safety needle 20 is an improved needle for use with a typical syringe (not shown). Safety needle 20 is provided with a slidable sleeve 22. Referring to FIG. 1, sleeve 22 is shown in an initial position in which the sharp tip 26 of needle 28 is exposed by the end 24 of sleeve 22. The sleeve 22 is free to move rearwardly against the spring 42 bias, but is prevented from moving towards the sharp needle tip 26. When safety needle 20 is being used, needle tip 26 is inserted into the patient, causing sleeve end 24 to make contact with the patient's skin. As the needle insertion continues, sleeve 22 retracts towards the position of FIG. 2. In this position, sleeve 22 remains in contact with the patient, thus needle 28 is not exposed. Once safety needle 20 has been used and is being removed from the patient, sleeve 22 slides back to a fully extended position. Sleeve 22 extends outwardly beyond the initial position of FIG. 1 into the locked position shown in FIG. 3, thus preventing a person from being accidentally stuck by a used needle 28 and also preventing safety needle 20 from being reused.

For particular applications where the patient may have to self-inject, or where the patient is needle-phobic, the design can be adapted to permit the needle sleeve in its initial position to entirely cover and hide the needle. The needle sleeve can also be manufactured from an opaque material.

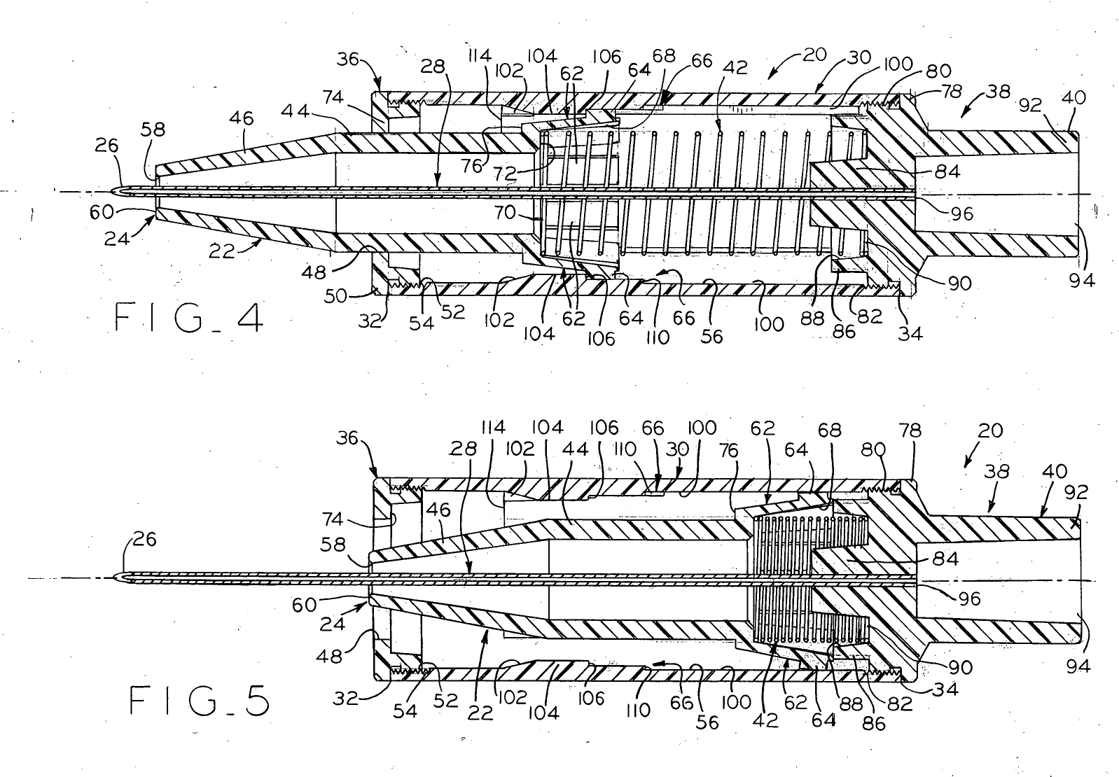

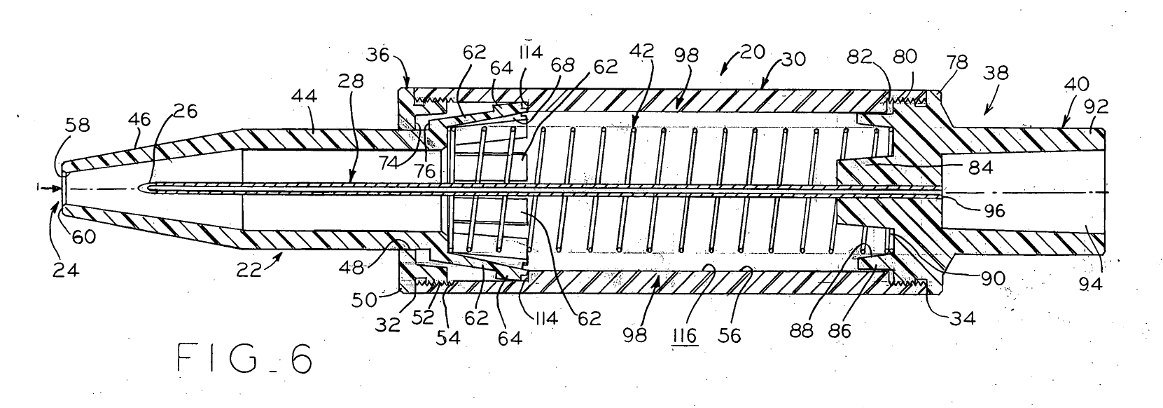

Referring to FIGS. 4, 5, 6, and 7, safety needle 20 is shown, respectively, in initial, retracted and locked positions. Safety needle 20 includes exterior casing 30 in which sleeve 22 is slidably mounted. Casing 30 is substantially cylindrical having open ends 32 and 34. Collar 36 is secured within end 32 to close end 32 and to capture sleeve 22 within casing 30. Secured to the opposite end 34 of casing 30 is needle mount 38 in which needle 28 is fixedly mounted. Collar 36 and needle mount 38 are threadedly secured in ends 32 and 34 of casing 30, respectively; however, collar 36 and needle mount 38 may be secured to casing 30 by any suitable method. Needle mount 38 is also provided with receiving end 40 for engagement with a syringe (not shown). Located within casing 30 is spring 42 which engages both needle mount 38 and sleeve 22 to bias sleeve 22 into its initial position of FIG. 4 and to return sleeve 22 to its extended locked position of FIG. 6 after sleeve 22 has been retracted.

Sleeve 22 has central main body portion 44 positioned primarily within casing 30 and tapered end portion 46 extending outwardly from the main body portion. Tapered end portion 46 passes through collar 36 when safety needle 20 is assembled to cover all but the sharp tip 26 of the needle 28 as shown in FIG. 4. When mounted in casing 30, tapered end portion 46 and at least a portion of main body portion 44 of sleeve 22 passes through opening 48 in collar 36. Collar opening 48 has a diameter but somewhat greater than the outer diameter of main body portion 44 whereby sleeve body portion 44 can move slidably relative to collar 36.

Collar 36 further includes flanged portion 50 which engages end 32 of casing 30, and threads 52 which engage threads 54 formed in casing inner wall 56. Any other suitable means of attachment between collar 36 and casing 30 may be utilized. Opening 58 is defined in end 60 of tapered end portion 46 through which end 26 of needle 28 passes when safety needle 20 is used. Tapered end opening 58 is large enough to allow passage of needle 28, but small enough to prevent a person from sticking a finger into tapered end portion 46 and contacting needle 28.

The opposite end of sleeve main body portion 44 is provided with a plurality of resilient fingers 62. Fingers 62 are integrally formed about the upper end of main body portion 44. Fingers 62 extend outwardly from main body portion 44 at an angle relative to the central longitudinal axis of body portion 44 and engage inner wall 56 of casing 30 as will be described further hereinbelow. Fingers 62 are resilient and can be flexed inwardly toward the central longitudinal axis of sleeve 22. At least one of fingers 62 is provided with projection 64 which follows track system 66 integrally formed on casing inner wall 56 to facilitate locking of sleeve 22 in its extended position after use of safety needle 20. Fingers 62 further define cup-shaped cavity 68 in which end 70 of spring 42 is received for applying a biasing force against bottom 72 of cavity 68. Edge 74 of collar opening 48 may be engaged by ledge 76 defined by fingers 62 if sleeve 22 moves out of casing 30 beyond its locked position of FIG. 6 thus preventing sleeve 22 from exiting from casing 30.

Needle mount 38 is threadedly secured to end 34 of casing 30 by means of flanged portion 78 which engages casing end 34 and threads 80 which engage threads 82 formed in casing inner wall 56. Any other suitable means of attachment between needle mount 38 and casing 30 may be utilized. Needle mount 38 includes central portion 84 through which needle 28 extends and is fixedly mounted by any suitable method. Positioned in surrounding relationship of central portion 84 is annular wall 86 which together with central portion 84 defines cavity 88 in which end 90 of spring 42 is received. Central portion 84 extends into the center of spring 42 when safety needle 20 is assembled. Needle mount 38 is also provided with receiving end 40 which is extends outwardly from engagement with casing 30. Receiving end 40 includes annular wall 92 which defines cavity 94 to which end 96 of needle 28 extends. A syringe is secured to receiving end 40 of safety needle 20 such that needle 28 is in fluid communication with the syringe.

The components of safety needle 20 are constructed from any suitable medical grade materials able to withstand sterilization and use. Sleeve 22, casing 30, collar 36 and needle mount 38 may be constructed from plastic materials, for example, by any suitable method including injection molding, or the like. Needle 28 is a conventional needle formed from medical grade stainless steel or other suitable material. Spring 42 is a conventional spring which may be formed from plastic or metal materials able to withstand the biasing forces exerted during use of safety needle 20.

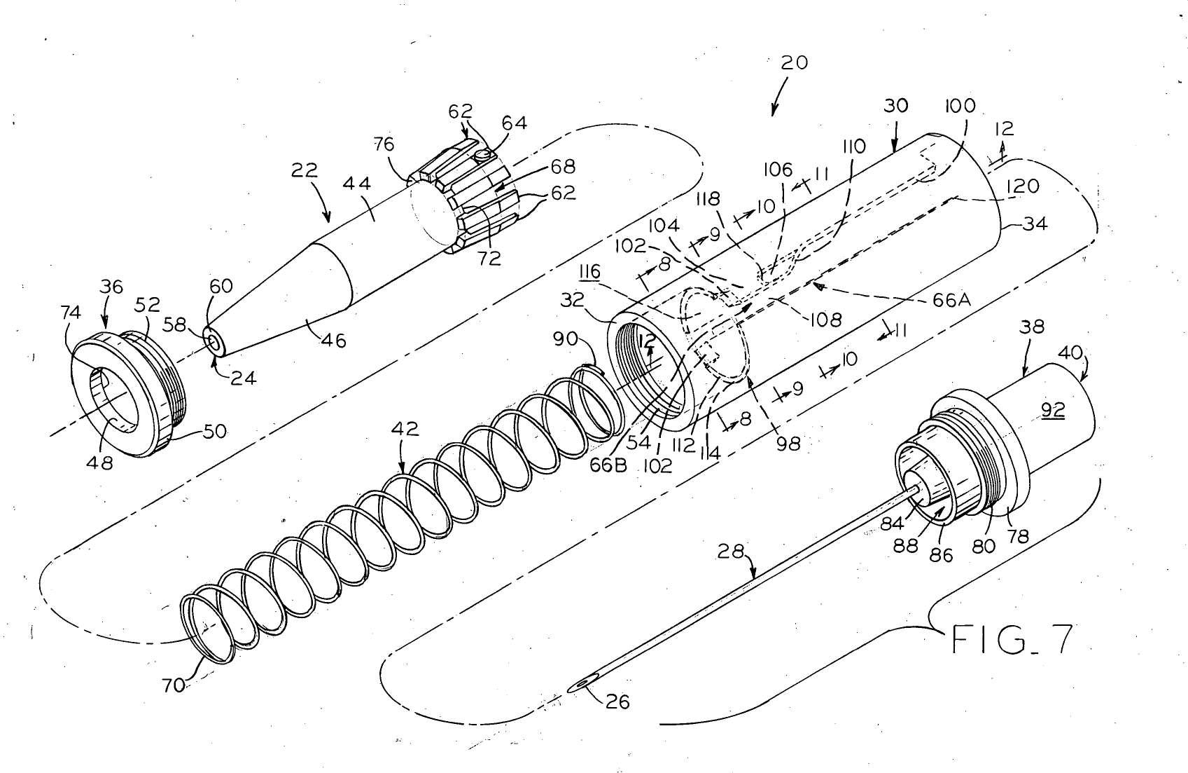

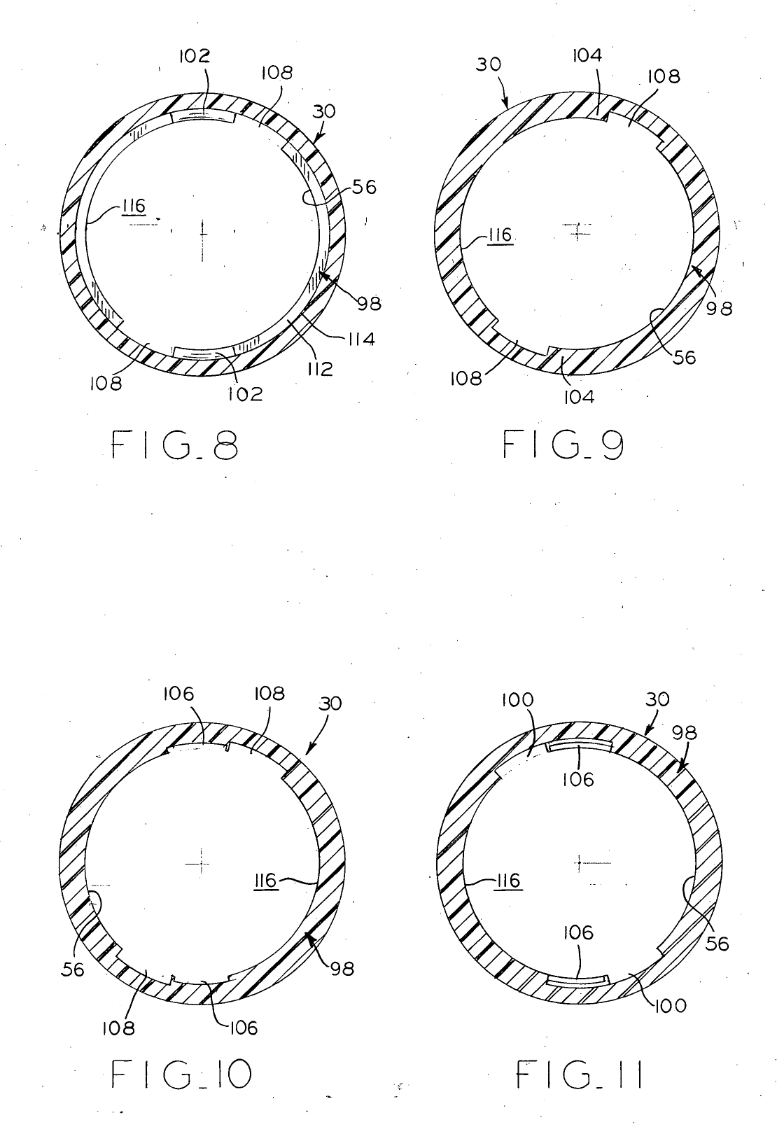

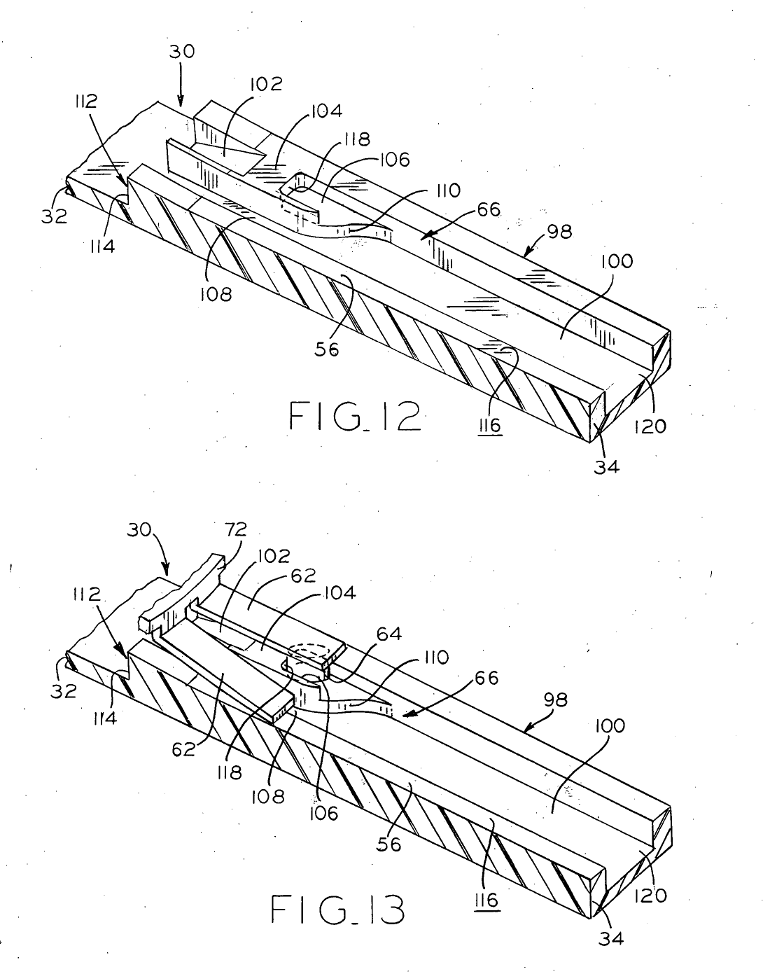

Referring to FIGS. 7-17, inner wall 56 of casing 30 includes a track system 66 for facilitating locking of sleeve 22. Track system 66 includes two diametrically opposed track systems 66A and 66B located on radially opposite sides of casing 30, each system being engaged by one projection 64 on finger 62. Track systems 66A and 66B are each defined by a thickened wall portion 98 which extends along a substantially, longitudinal portion of casing 30. Formed in thickened wall portion 98 is longitudinally extending passageway 100. Also included are installation ramps 102 which are inclined toward plateau 104. Located on the opposite side of plateau 104 is recessed cavity 106 in which projection 64 is received upon assembly of sleeve 22 into casing 30. The width of passage 100 from recessed cavity 106 toward end 34 of casing 30 is approximately twice that of passage portion 108 located adjacent plateau 104. The decrease in width of passage portion 108 in thickened wall portion 98 defines a cam profile 110 which directs projection 64 toward the locked position during use of safety needle 20. Edge 112 of thickened wall portion 98 is undercut slightly at 114 to provide a locking interference fit with the tips of fingers 62 as will be discussed hereinbelow.

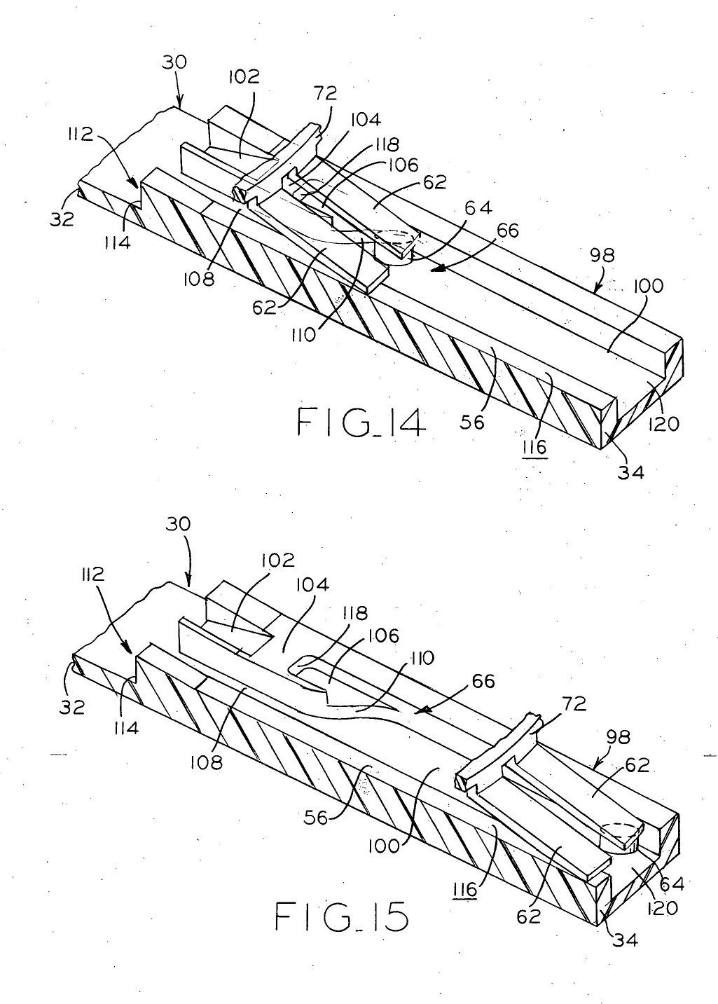

During assembly of safety needle 20, sleeve 22 is assembled with casing 30, and resilient fingers 62 with projections 64 are aligned with installation ramps 102, by the use of an assembly tool which guides the fingers with projections onto the ramps in the casing and also deflects the remaining fingers inwardly towards the main axis such that they may bypass the locking feature. Projections 64 slide across plateau 104 and are received in recessed cavities 106 (FIG. 13). Collar 36 is then threadedly secured to casing 30 and spring 42 is positioned in casing 30 with end 70 received in cup-shaped cavity 68. Needle 28 is then passed through spring 42 and into sleeve 22 while aligning end 90 of spring 42 with cavity 88 defined in needle mount 38. Needle mount 38 is then secured to end 34 of casing 30, compressing spring 42 and biasing projections 64 into contact with edge 118 of recessed cavity 106. As assembled, safety needle 20 is in the initial position shown in FIGS. 1 and 4 with sleeve 22 exposing end 26 of needle 28 prior to use.

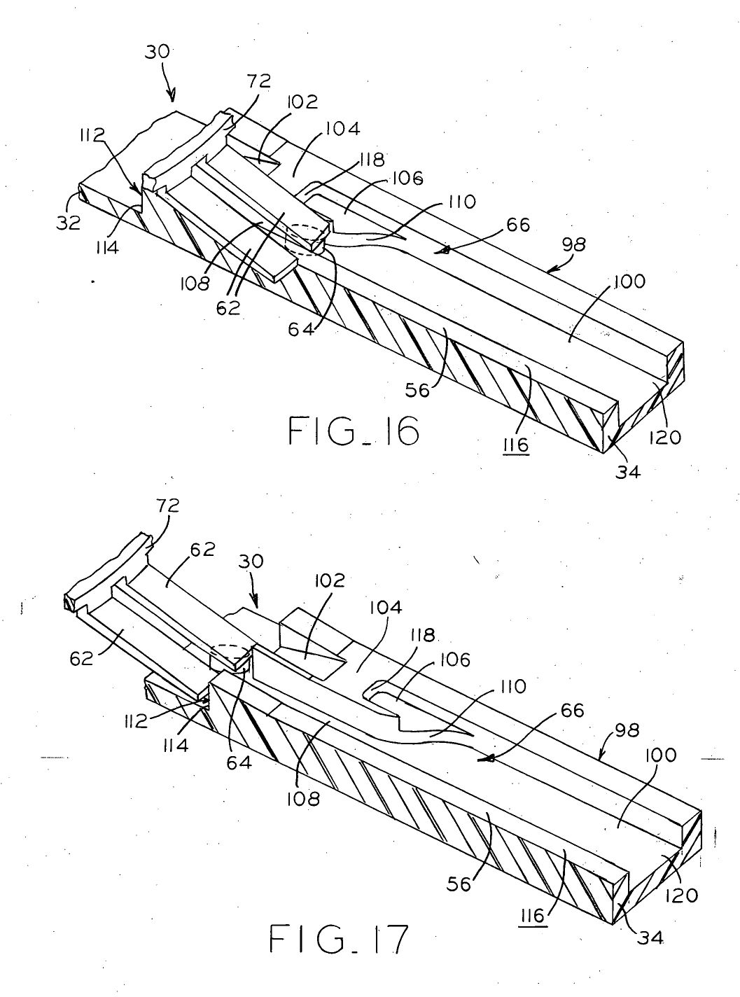

During operation of safety needle 20, projections 64 travel along track system 66 to guide sleeve 22 into a locked position. The safety needle 20 is placed against the skin of the patient with needle 28 penetrating the skin of the patient. Tapered end portion 46 of sleeve 22 then makes contact with the patient. End 60 of sleeve 22 remains in contact with the patient during the injection such that needle 28 is not exposed. At this point, sleeve 22 is in a retracted position shown in FIGS. 2 and 5. During the injection, projections 64 slide out of recessed cavities 106, dropping off thickened wall portion 98 and moving toward end 120 of longitudinal passageway 100 as shown in FIGS. 14 and 15. As needle 28 is removed from the patient, sleeve 22 begins to return to the extended position of FIGS. 3 and 6 due to the biasing force of spring 42. As sleeve 22 extends from casing 30, projections 64 slide along passageway 100. The projections 64 contact cam profile 110 which directs projections 64 into passage portion 108 as shown in FIG. 16. As projections 64 follow cam profile 110, sleeve 22 rotates in a clockwise direction so as to pass through passage portion 108. However, it should be noted that, by using the appropriate track system configuration, rotation can be arranged in either the clockwise or counterclockwise direction. Sleeve 22 continues to extend from casing 30 until fingers 62 fall off of thickened wall portion 98 and flex outwardly to become engaged with undercut portion 114 as shown in FIG. 17. The engagement between fingers 64 and undercut portion 114 locks the position of sleeve 22 thus preventing sleeve 22 from returning to its retracted position and exposing needle 28.

It should be noted that, while the fingers 62 have been shown as formed integrally with sleeve 22, and the track system 66 with casing 30, it is also possible that the fingers could be formed as part of casing 30 and the track could be part of sleeve 22.

While this invention has been described as having exemplary designs, the present invention may be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

1. An accessory device for use with a medical apparatus in which fluid is drawn or expelled through a hollow needle, said device comprising:
an elongated hollow body;
a needle connected to said hollow body;
a sleeve surrounding said needle, said sleeve reciprocally, movably associated with said hollow body for movement into and out of said hollow body to thereby expose more or less of the length of said needle, said sleeve having a first position wherein said sleeve covers the major portion of the length of said needle, a second position wherein a sufficient portion of the length of said needle is exposed so that it is available for use in a medical procedure, and a third position wherein said sleeve covers said entire needle and is locked in position to prevent exposure of any part of said needle; and
one of said sleeve and said body including a track and the other of said sleeve and said body including a stop member, said stop member operatively guidedly associated with said track whereby said sleeve can move from said first position to said second position and thereafter to said third position and said stop member locks said sleeve in said third position.

2. The device according to claim 1 wherein said stop member comprises a flexible member.

3. The device according to claim 2 wherein said sleeve includes said flexible member.

4. The device according to claim 2 wherein said sleeve includes a plurality of flexible members.

5. The device according to claim 1 wherein said body includes a plurality of tracks.

6. The device according to claim 2 wherein said flexible member includes a projection which engages with said track whereby said track guides said flexible member.

7. The device according to claim 1 wherein said track includes a first portion to permit said sleeve to move from said first to said second position, and a second portion to permit said sleeve to move from said second position to said third position.

8. The device according to claim 1 wherein said body includes a stop, said flexible member engaging said stop when said sleeve reaches said third position.

9. The device according to claim 2 wherein said flexible member can move radially inwardly and outwardly relative to the central axis of said longitudinal body.

10. The device according to claim 1 further comprising a biasing spring for biasing said sleeve into said first position.

11. The device according to claim 1 further including a collar operatively associated with said body and sleeve for preventing said sleeve from becoming disassociated with said body.

12. An accessory device for use with a medical apparatus in which fluid is drawn or expelled through a hollow needle, said device comprising:
an elongated hollow body;
a needle connected to said hollow body;
a sleeve surrounding said needle, said sleeve reciprocally, movably associated with said hollow body for movement into and out of said hollow body to thereby expose more or less of the length of said needle, said sleeve having a first position wherein said sleeve covers a major portion of the length of said needle, a second position wherein a sufficient portion of the length of said needle is exposed and available for use in a medical procedure, and a third position wherein said sleeve covers said entire needle and is locked in position to prevent exposure of any part of said needle; and
said sleeve including a flexible finger and said body including a track, said flexible finger operatively guidedly associated with said track whereby said sleeve can move from said first position to said second position and thereafter to said third position and said flexible finger locks said sleeve in said third position.

13. The device according to claim 12 wherein said sleeve includes a plurality of flexible fingers and wherein said body includes a plurality of tracks.

14. The device according to claim 12 wherein said flexible finger includes a projection which engages with said track whereby said track guides said flexible finger.

15. The device according to claim 12 wherein said track includes a first portion to permit said sleeve to move from said first to said second position, and a second portion to permit said sleeve to move from said second position to said third position.

16. The device according to claim 12 wherein said flexible finger engages said stop when said sleeve reaches said third position.

17. The device according to claim 12 wherein said flexible finger can move radially inwardly and outwardly relative to the central axis of said longitudinal body.

18. The device according to claim 12 further comprising a biasing spring for biasing said sleeve into said first position.

19. The device according to claim 12 further including a collar operatively associated with said body and sleeve for preventing said sleeve from becoming disassociated with said body.
